(19) United States
(12) Patent Application Publication
(10) Pub. No.: US 2012/0142632 A1
(43) Pub. Date: Jun. 7, 2012

(54) DIAGNOSTIC AND PROGNOSTIC ASSAY

(75) Inventor: DUNCAN JOHN CAMPBELL, CARLTON NORTH (AU)

(73) Assignee: ST. VINCENT'S INSTITUTE OF MEDICAL RESEARCH, FITZROY (AU)

(21) Appl. No.: 13/198,115

(22) Filed: Aug. 4, 2011

Related U.S. Application Data
(60) Provisional application No. 61/370,767, filed on Aug. 4, 2010.

(51) Int. Cl. A61K 31/727; A61P 9/00; G06F 19/00; G01N 33/566

(52) U.S. Cl. 514/56; 436/501; 435/7.92; 435/7.1; 702/19

(57) ABSTRACT

The present disclosure relates generally to the field of diagnostic and prognostic assays for diseases and conditions of the systemic vasculature. The present disclosure teaches an assay for identifying such a disease or condition of the systemic vasculature as well as classifying and determining the state or stage of the disease or condition or the risk of developing the disease or condition. The assay enabled herein is also useful in the stratification of a subject with respect to a risk of developing various diseases and conditions of systemic vasculature. The assay taught herein is also capable of integration into pathology architecture to provide a diagnostic and reporting system.

### FILING DATA

This application is associated with and claims priority from U.S. Provisional Patent Application No. 61/370,767, filed on 4 Aug. 2010, entitled “Diagnostic and prognostic assay”, the entire contents of which, are incorporated herein by reference.

### FIELD

The present disclosure relates generally to the field of diagnostic and prognostic assays for diseases and conditions of the systemic vasculature. The present disclosure teaches an assay for identifying such a disease or condition of the systemic vasculature as well as classifying and determining the state or stage of the disease or condition or the risk of developing the disease or condition. The assay enabled herein is also useful in the stratification of a subject with respect to a risk of developing various diseases and conditions of systemic vasculature. The assay taught herein is also capable of integration into pathology architecture to provide a diagnostic and reporting system.

### BACKGROUND

Bibliographic details of references provided in the subject specification are listed at the end of the specification.

Reference to any prior art is not, and should not be taken as an acknowledgment or any form of suggestion that this prior art forms part of the common general knowledge in any country.

Of the diseases affecting the systemic vasculature, coronary artery disease remains one of the major causes of premature mortality and morbidity (Lloyd-Jones et al., Circulation 121:948-954, 2010). Whereas some patients with coronary artery disease experience chronic angina without evident myocardial infarction (MI), other patients may experience MI as the initial symptom of coronary artery disease or after a period of angina.

Myocardial ischemia and infarction occur when myocardial oxygen demand exceeds supply. Whether flow-limiting coronary lesions produce myocardial ischemia or infarction depends on whether myocardial perfusion is reduced below the level required to maintain viability. Among all organs, the heart is unique in that oxygen extraction is constantly close to maximal (Messer et al., J Clin Invest 41:725-742, 1962) and the importance of the capillary bed to the ischemic vulnerability of the hypertrophic myocardium is well recognized (Anversa and Sonneblick Prog Cardovasc Dis 33:49-70, 1990). MI is typically the result of an abrupt reduction in coronary flow due to plaque rupture or erosion with thrombus formation and/or distal embolization of thrombus and plaque material (Naghavi et al., Circulation 108:1664-1672, 2003; Heusch et al., Circulation 120:1822-1836, 2009). Whereas complex coronary plaques increase the risk of MI (Goldstein et al., N Engl J Med 343:915-922, 2000), coronary collaterals protect ischemic myocardium from infarction (Koerselman et al., Circulation 107:2507-2511, 2003; Berry et al., Eur Heart K 28:278-291, 2007; van Royen et al., J Am Coll Cardiol 55:17-25, 2009).

There is a need to identify additional risk factors for individuals at risk of MI or other diseases or adverse conditions of the systemic vasculature.

### SUMMARY

The present disclosure teaches that reduced vascular density is a contributing factor toward the development or progression of diseases or conditions of the systemic vasculature. Such diseases and conditions include heart disease, stroke and organ damage due to reduced blood flow. In particular, it is proposed herein that reduced microvascular density contributes to reduced organ vasodilator reserve in the tissue and such tissue including myocardium is proposed to more likely suffer damage from an ischemic condition or event.

As disclosed herein the levels of one or more advanced glycation end products (AGEs) and angiogenesis factors or the ratios of levels of two or more “AGE” and/or angiogenesis factors and/or the level of microvascular density are instructive in the assessment of vascular density which in turn determines the presence, stage of development, risk of development and/or severity of a disease or condition or event of the systemic vasculature.

An association is, therefore, identified between the level or ratio of AGEs and angiogenesis factors in circulatory fluid in a subject and level of microvascular density and risk of development of a disease, condition or event of the systemic vasculature. The terms AGE and “angiogenesis factor” include a biomarker, indicator or an analyte. By “microvascular density” includes capillary length density and diffusion radius. By “disease or condition or event of the systemic vasculature” means a vascular disease such as heart disease or cardiovascular disease and its various manifestations such as myocardial infarction (MI) and atherosclerosis as well as stroke and other conditions arising from reduced arterial or venous blood flow including a reduction in blood flow leading to organ damage. The term “heart disease” includes an individual condition as well as a collection of conditions within the clinical spectrum of symptomatic or asymptomatic heart disease.

Reference to “heart disease”, therefore, includes conditions such as coronary heart disease (including angina pectoris and myocardial infarction (MI)), atherosclerosis, cardiomyopathy (including that associated with arrhythmia), cardiovascular disease, ischaemic heart disease, heart failure (including cor pulmonale), hypertensive heart disease (including left ventricular hypertrophy and congestive heart failure), inflammatory heart disease (including endocarditis, inflammatory cardiomegaly and myocarditis) and valvular heart disease (including aortic valve stenosis and mitral valve prolapse). Heart disease spectrum also includes associated conditions such as aortic aneurysm, hypertension, thrombosis and pericarditis. Heart disease is a spectrum of clinical manifestations. In accordance with the present disclosure, reduced coronary microvascular density is proposed to contribute to reduced coronary vasodilator reserve of myocardium and to the vulnerability to ischemic damage.

The AGEs and angiogenesis factors and in particular their levels and/or ratios and the level of microvascular density are proposed to provide a risk indicator of reduced vascular density leading to vascular disease progression and degree of severity and allows a classification of the vascular disease. This risk ranges from minor to extreme. Knowledge of the level of risk enables intervention to mitigate further development of the vascular disease. The ability to monitor and identify markers of vascular disease including diagnosing a disease, condition or event in asymptomatic subjects further enables decisions on the type of medical intervention required from behavioural modification and medicaments to surgical intervention. The AGEs and angiogenesis factors and microvascular density are also instructive as to the level of risk for an individual developing more severe symptomology associated with vascular disease.

The present disclosure enables the determination that subjects with vascular disease or who are at risk of developing vascular disease exhibit altered levels or ratios of particular AGEs and angiogenesis factors and/or altered levels of microvascular density and these provide an indicator of the level or state of luminal density. Luminal density in turn provides an indicator as to the level of risk, state or classification of vascular disease or adverse condition such as heart disease or stroke, especially in symptomatic and asymptomatic subjects. By “classification” includes identifying subjects as vulnerable or non-vulnerable subjects. Hence, taught herein is the stratification of subjects into risk categories, treatment categories and likely progression outcomes. The term “luminal density” includes vascular density.

The AGE and angiogenesis factors contemplated herein include:

(i) carboxymethyl lysine (CML);

(ii) Vascular Endothelial Growth Factor A (VEGFA);

(iii) Vascular Endothelial Growth Factor Receptor-1 (VEGFR-1);

(iv) Low molecular weight fluorophore (LWF);

(v) Angiopoietin-2;

(vi) Tie-2;

(vii) Endostatin;

(viii) Placental growth factor (PGF); and

(ix) Hepatocyte growth factor (HGF).

The levels or ratios of levels of the AGE and angiogenesis factors and the level of microvascular density are determined relative to a control. The assay may also be automated or semi-automated. In particular, the levels or ratios of levels may be used as input data for multivariate or univariate analysis leading to an algorithm which can be used to generate an index of probability of having or progressing with a vascular disease.

The levels of the AGE and angiogenesis factors and microvascular density may also be used in combination with other standard indicators of vascular disease, whether biochemical markers, symptoms or electrocardial techniques.

Accordingly, one aspect of the present disclosure contemplates an assay to stratify a subject with respect to vascular disease, the assay comprising determining:

(A) the level or ratio of levels of an AGE and/or angiogenesis factor selected from the list consisting of:
(i) CML;
(ii) VEGFA;
(iii) VEGFR-1;
(iv) Low molecular weight fluorophore (LWF);
(v) Angiopoietin-2;
(vi) Tie-2;
(vii) Endostatin;
(viii) Placental growth factor (PGF); and
(ix) Hepatocyte growth factor (HGF); and/or

(B) the level of microvascular density;

wherein the level or ratio of the AGE and/or angiogenesis factor(s) and/or level of microvascular density relative to a control provides a correlation as to the presence, state, classification or progression of vascular disease. Ratios include the ratios of levels of angiopoietin-2:Tie-2 and VEGFR-1:VEGFA.

Particularly, the present disclosure teaches an assay to stratify a subject with respect to heart disease, the assay comprising determining:

(A) the level or ratio of levels of an AGE and/or angiogenesis factor selected from the list consisting of:
(i) CML;
(ii) VEGFA;
(iii) VEGFR-1;
(iv) Low molecular weight fluorophore (LWF);
(v) Angiopoietin-2;
(vi) Tie-2;
(vii) Endostatin;
(viii) Placental growth factor (PGF); and
(ix) Hepatocyte growth factor (HGF); and/or

(B) the level of microvascular density;

wherein the level or ratio of the AGE and/or angiogenesis factor(s) and/or level of microvascular density relative to a control provides a correlation as to the presence, state, classification or progression of heart disease.

Particularly, the present disclosure contemplates an assay to stratify a subject with respect to myocardial infarction (MI), the assay comprising determining:

(A) the level or ratio of levels of an AGE and/or angiogenesis factor selected from the list consisting of:
(i) CML;
(ii) VEGFA;
(iii) VEGFR-1;
(iv) Low molecular weight fluorophore (LWF);
(v) Angiopoietin-2;
(vi) Tie-2;
(vii) Endostatin;
(viii) Placental growth factor (PGF); and
(ix) Hepatocyte growth factor (HGF); and/or

(B) the level of microvascular density;

wherein the level or ratio of the AGE and/or angiogenesis factor and/or microvascular density relative to a control provides a correlation as to the presence, state, classification or progression of MI.

Another aspect taught therein is an assay to identify a subject with vascular disease, the assay comprising determining:

(A) the level or ratio of levels of an AGE and/or angiogenesis factor selected from the list consisting of:
(i) CML;
(ii) VEGFA;
(iii) VEGFR-1;
(iv) Low molecular weight fluorophore (LWF);
(v) Angiopoietin-2;
(vi) Tie-2;
(vii) Endostatin;
(viii) Placental growth factor (PGF); and
(ix) Hepatocyte growth factor (HGF); and/or

(B) the level of microvascular density;

wherein the level or ratio of the AGE and/or angiogenesis factor(s) and/or microvascular density relative to a control provides an indication of the presence or absence of vascular disease.

Yet another aspect enabled herein an assay to stratify a subject as a vulnerable or non-vulnerable subject with respect to the risk of developing heart disease, the assay comprising determining:

(A) the level or ratio of an AGE and/or angiogenesis factor selected from the list consisting of:
(i) CML;
(ii) VEGFA;
(iii) VEGFR-1;
(iv) Low molecular weight fluorophore (LWF);
(v) Angiopoietin-2;
(vi) Tie-2;
(vii) Endostatin;
(viii) Placental growth factor (PGF); and
(ix) Hepatocyte growth factor (HGF); and/or

(B) the level of microvascular density;

wherein the level or ratio of the AGE and/or angiogenesis factor(s) and/or microvascular density relative to a control identifies the subject as being vulnerable or non-vulnerable.

Still another aspect of the present disclosure contemplates the use of a panel of AGE and/or angiogenesis factors selected from the list consisting of:

(i) CML;

(ii) VEGFA;

(iii) VEGFR-1; and/or

(iv) Low molecular weight fluorophore (LWF);

(v) Angiopoietin-2;

(vi) Tie-2;

(vii) Endostatin;

(viii) Placental growth factor (PGF); and

(ix) Hepatocyte growth factor (HGF); and/or

in the manufacture of an assay to identify the presence, state, classification or progression of vascular disease in a subject.
In an embodiment, the present disclosure teaches an assay to stratify a subject with cardiovascular disease (CAD) with respect to the risk of the subject developing a myocardial infarction (MI), the assay comprising:
(i) selecting a subject having symptoms of CAD or who is at risk of developing CAD; and
(ii) determining the plasma levels of an advanced glycation end (AGE) product or angiogenesis factor selected from the list consisting of carboxymethyl lysine (CML); low molecular weight fluorophore (LMWF); vascular endothelial growth factor-A (VEGF-A); vascular endothelial growth factor receptor-1 (VEGFR-1); angiopoietin-2: Tie-2; endostatin; placental growth factor (PLGF); and hepatocyte growth factor (HGF);
   wherein the subject is stratified as having a MI or is at risk of developing a MI when:
(i) levels of one or more of CML, LMWF, VEGF-A, and/or Tie-2 are reduced compared to a control not having a MI; and/or
(ii) levels of one or more of VEGFR-1, VEGFR-1:VEGFA ratio, angiopoietin-2, angiopoietin-2:Tie-2 ratio, endostatin, PLGF and/or HGF are elevated compared to a control not having a MI.

The present disclosure teaches the use of microvascular density in the manufacture of an assay to identify the presence, state, classification or progression of vascular disease in a subject.

Yet another aspect taught herein is a method for diagnosing the likelihood of a subject exhibiting reduced coronary microvascular density and thereby at risk of having reduced coronary vasodilator reserve of myocardium and at increased risk of myocardial infarction, the method comprising determining:

(A) levels or ratio of levels of CML, LMWF, VEGF-A, and/or Tie-2 are reduced compared to a control not having a MI; and/or

(B) the level of microvascular density;

wherein:
(i) levels of one or more of CML, LMWF, VEGF-A, and/or Tie-2 are reduced compared to a control not having a MI; and/or
(ii) levels of one or more of VEGFR-1, VEGFR-1:VEGFA ratio, angiopoietin-2, angiopoietin-2:Tie-2 ratio, endostatin, PLGF and/or HGF are elevated compared to a control not having a MI.

Another aspect taught herein is a method for diagnosing the likelihood of a subject exhibiting reduced coronary microvascular density and thereby at risk of having reduced coronary vasodilator reserve of myocardium remote from the site of an infarction, the method comprising:
(i) selecting a subject having symptoms of cardiovascular disease (CAD) or who is at risk of developing CAD; and
(ii) determining the levels of an advanced glycation end (AGE) product or angiogenesis factor selected from the list consisting of carboxymethyl lysine (CML); low molecular weight fluorophore (LMWF); vascular endothelial growth factor-A (VEGF-A); vascular endothelial growth factor receptor-1 (VEGFR-1); angiopoietin-2: Tie-2; endostatin; placental growth factor (PLGF) and hepatocyte growth factor (HGF);
   wherein the subject is stratified as having reduced coronary microvascular density or is at risk of developing same when:
(i) levels of one or more of CML, LMWF, VEGF-A, and/or Tie-2 are reduced compared to a control not having a MI; and/or
(ii) levels of one or more of VEGFR-1, VEGFR-1:VEGFA ratio, angiopoietin-2, angiopoietin-2:Tie-2 ratio, endostatin, PLGF and/or HGF are elevated compared to a control not having a MI.

Another aspect enabled herein a method of treatment or prophylaxis of a subject comprising assaying the subject with respect to vascular disease by determining:

(A) the level or ratio of levels of an AGE and/or angiogenesis factor selected from the list consisting of:
(i) CML;
(ii) VEGFA;
(iii) VEGFR-1;
(iv) Low molecular weight fluorophore (LWF);
(v) Angiopoietin-2;
(vi) Tie-2;
(vii) Endostatin;
(viii) Placental growth factor (PGF); and
(ix) Hepatocyte growth factor (HGF); and/or

(B) the level of microvascular density;

wherein the level or ratio of the AGE and/or angiogenesis factor(s) and/or microvascular density relative to a control provides a correlation to the presence, state, classification or progression of vascular disease and then providing therapeutic and/or behavioural modification to the subject.

In an embodiment, the present disclosure teaches a method of treatment or prophylaxis of a subject with cardiovascular disease (CAD), the method comprising stratifying the subject with respect to the risk of the subject developing a myocardial infarction (MI), the stratification comprising:
(i) selecting a subject having symptoms of CAD or who is at risk of developing CAD; and
(ii) determining the levels of an advanced glycation end (AGE) product or angiogenesis factor selected from the list consisting of carboxymethyl lysine (CML); low molecular weight fluorophore (LMWF); vascular endothelial growth factor-A (VEGF-A); vascular endothelial growth factor receptor-1 (VEGFR-1); angiopoietin-2: Tie-2; endostatin; placental growth factor (PLGF); and/or hepatocyte growth factor (HGF); and/or
   wherein the subject is stratified as having a MI or is at risk of developing a MI when:
(i) levels of one or more of CML, LMWF, VEGF-A, and/or Tie-2 are reduced compared to a control not having a MI; and/or
(ii) levels of one or more of VEGFR-1, VEGFR-1:VEGFA ratio, angiopoietin-2, angiopoietin-2:Tie-2 ratio, endostatin, PLGF and/or HGF are elevated compared to a control not having a MI; and then providing a therapeutic or behavoral modification to mitigate the risk of the MI.

The “stratification” is in effect a level of risk that a subject has vascular disease or is developing vascular disease or is likely to develop symptoms of vascular disease.

The determination of the levels or ratios of the AGE and/or angiogenesis factors and/or level of microvascular density may also be used in combination with other indicators of vascular disease and may also be used to monitor efficacy of treatment. In addition, the assay may be useful in determining the most effective therapeutic or behavioural intervention to treat vascular disease in symptomatic or asymptomatic subjects. As indicated above, reference to “vascular disease” includes any disease or adverse condition of the systemic vasculature such as the spectrum of heart disease including MI and stroke. An angiogenesis factor includes an advanced glycation end-product (AGE-product) related factor. It is proposed herein that subjects with heart disease or who are at risk of developing heart disease have (i) levels of one or more of CML, LMWF, VEGF-A, and/or Tie-2 are reduced compared to a control not having a MI; and/or (ii) levels of one or more of VEGFR-1, VEGFR-1:VEGFA ratio, angiopoietin-2, angiopoietin-2:Tie-2 ratio, endostatin, PLGF and/or HGF are elevated compared to a control not having a MI.

The assay may also be used in a personalized medicine approach in the management of vascular disease and/or as part of a pathology architecture platform.

In an embodiment, microvascular density is not determined and only one or more of CML, LMWF, VEGFA, VEGFR-1, angiopoietin-2, Tie-2, endostatin, PLGF and/or HGF is determined

### BRIEF DESCRIPTION OF THE FIGURES

FIGS. 1A through H are graphical representations showing capillary length density of no myocardial infarction (no-MI) and non-ST-segment-elevation myocardial infarction (NSTEMI) patients, presented separately for men (Panel A) and women (Panel B), patients with diabetes (Panel C), or metabolic syndrome without diabetes (Panel D), and presence or absence of occluded coronary artery (Panels E and F) or wall motion abnormality (Panels G and H). Data shown as means±SEM.

FIGS. 2A through C are graphical representations of the correlation between myocardial capillary length density and time between NSTEMI and surgery (Panel A), maximum plasma troponin I levels for NSTEMI patients (Panel B), and plasma NT-proBNP levels of NSTEMI patients before surgery (Panel C). The correlation coefficients were from a univariate Pearson's correlation analysis.

FIGS. 3A through D are graphical representations of the effect of duration and character of angina before surgery on myocardial capillary length density of no-MI patients (Panels A and B) and of NSTEMI patients before myocardial infarction (Panels C and D). Unstable angina refers to angina of recent onset (<2 months) or increasing angina (angina of duration >2 months that was increasing in frequency and/or severity). For no-MI patients with unstable angina, 5 had angina of recent onset and 20 had increasing angina. For NSTEMI patients with unstable angina, 5 had angina of recent onset and 8 had increasing angina. Data shown as means±SEM; there were no statistically significant differences between the groups in any panel.

FIG. 4 is a diagrammatic representation of differences in arteriolar and capillary length density between no-MI patients (normal myocardium) and NSTEMI patients (vulnerable myocardium).

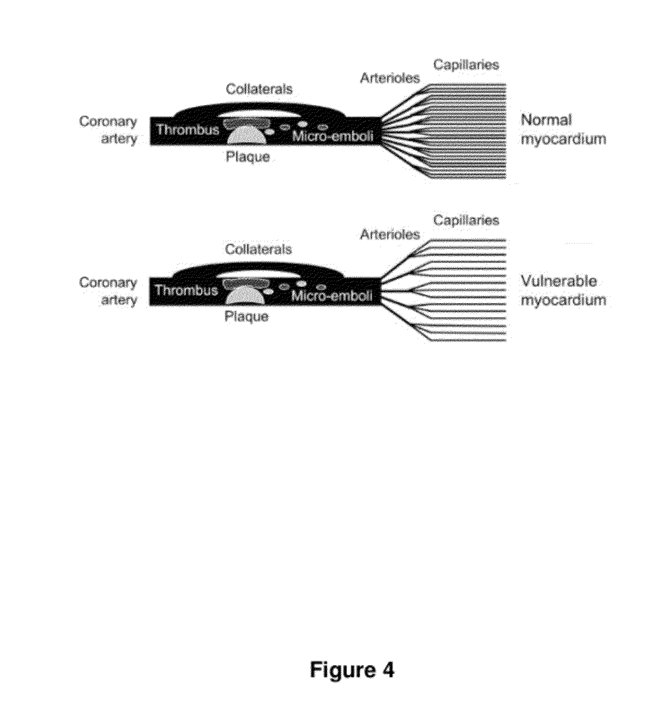

### DETAILED DESCRIPTION

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

As used in the subject specification, the singular forms “a”, “an” and “the” include plural aspects unless the context clearly dictates otherwise. Thus, for example, reference to “a factor” includes a single factor, as well as two or more factors; reference to “an ischemic condition” includes an ischemic condition or two or more ischemic conditions; reference to “the disclosure” includes single and multiple aspects taught by the disclosure; and so forth.

The use of numerical values in the various ranges specified in this specification, unless expressly indicated otherwise, are stated as approximations as though the minimum and maximum values within the stated ranges were both preceded by the word “about”. In this manner, slight variations above and below the stated ranges can be used to achieve substantially the same results as values within the ranges. Also, the disclosure of these ranges is intended as a continuous range including every value between the minimum and maximum values. In addition, the present disclosure teaches ratios of two or more markers providing a numerical value associated with a level of risk of vascular disease development or progression or presence.

A rapid, efficient and sensitive assay is provided for the stratification of a vascular disease in symptomatic and asymptomatic subjects.

“Stratification” includes identification, diagnosing, clarification, monitoring and/or determination of the presence, level, severity, state and/or classification of vascular disease. Generally, this is based on comparing a knowledge base of levels or ratios of angiogenesis factors in body fluid including plasma and whole blood and/or the level of microvascular density to another knowledge base of predetermined levels, statistically correlated to vascular disease or a condition or symptom within the spectrum of diseases or conditions of the systemic vasculature. The term “angiogenesis factor” includes AGE-related factors. “Microvascular density” includes capillary length density and diffusion radius. It is proposed herein that the level or ratio of AGE and/or angiogenesis factor(s) and level of microvascular density are instructive of reduced vascular density which contributes to reduced vasodilator reserve in tissue. In particular, the level or ratios of AGE and/or angiogenesis factor(s) and/or level of microvascular density enables a determination of whether the coronary microvascular density is reduced to a level so as to cause or facilitate reduced coronary vasodilator reserve of the myocardium. Even more particularly, myocardium with reduced vascular density is more likely to suffer ischemic damage. Furthermore, reduced vascular density is proposed herein to contribute mechanistically to ischemic damage. Without intending to limit the present disclosure to any one theory or mode of action, it is proposed that myocardium with reduced vascular density is less able to survive or escape damage from ischemic insult. This vulnerability to ischemic insult is due in part to reduced vasodilator reserve and increased diffusion radius.

Hence, the present disclosure identifies a correlation between the level or ratios of particular AGE and/or angiogenesis factor(s) and/or level of microvascular density in a subject and vascular disease. A disease or adverse condition or event of the systemic vasculature includes heart disease and all manifestations thereof such as cardiovascular disease, stroke and organ damage due to ischemic conditions. The term “heart disease” as used herein is to be considered as an individual condition as well as a spectrum of conditions including a range of risk indicators of the level of disease progression. This risk ranges from minor to extreme. The ability to monitor and identify markers of heart disease enables decisions on the type of medical intervention required from behavioural modification and medicaments to surgical intervention. This is particularly the case with asymptomatic individuals or those having a family history of heart disease.

The present disclosure particularly extends to any or all conditions within the clinical spectrum of “heart disease”.

Such conditions include, without being limited to, myocardial infarction (MI), cardiomyopathies, such as, alcoholic cardiomyopathy, coronary artery disease, congenital heart disease, nutritional diseases affecting the heart, ischemic (or ischaemic) cardiomyopathy, hypertensive cardiomyopathy, valvular cardiomyopathy, inflammatory cardiomyopathy, cardiovascular disease, such as atherosclerosis, ischaemic heart disease, heart failure, hypertensive heart disease, such as, left ventricular hypertrophy, coronary heart disease, (congestive) heart failure, hypertensive cardiomyopathy, cardiac arrhythmias, inflammatory heart disease, such as, endocarditis, inflammatory cardiomegaly, myocarditis, valvular heart disease, such as, aortic valve stenosis, mitral valve prolapse and valvular cardiomyopathy

Reference herein to a “subject” includes a human which may also be considered an individual, patient, host, recipient or target. The subject may also be an animal, such as used in an animal model. The term “angiogenesis factor” includes a analyte, marker, indicator, risk factor and the like.

The AGE and angiogenesis factors contemplated herein include:

(i) CML;

(ii) VEGFA;

(iii) VEGFR-1; and

(iv) Low molecular weight fluorophore (LWF);

(v) Angiopoietin-2;

(vi) Tie-2;

(vii) Endostatin;

(viii) Placental growth factor (PGF); and

(ix) Hepatocyte growth factor (HGF).

One or 2 or 3 or 4 or 5 or 6 or 7 or 8 or all 9 of the AGE angiogenesis factors may be determined

Accordingly, an aspect contemplated herein is an assay to stratify a subject with respect to vascular disease, the assay comprising determining:

(A) the level or ratio of levels of an AGE and/or angiogenesis factor selected from the list consisting of:
(i) CML;
(ii) VEGFA;
(iii) VEGFR-1;
(iv) Low molecular weight fluorophore (LWF);
(v) Angiopoietin-2;
(vi) Tie-2;
(vii) Endostatin;
(viii) Placental growth factor (PGF); and
(ix) Hepatocyte growth factor (HGF); and/or

(B) the level of microvascular density;

wherein the level or ratio of the AGE and/or angiogenesis factor(s) and/or level of microvascular density relative to a control provides a correlation as to the presence, state, classification or progression of vascular disease. A “ratio” includes the ratios of levels of angiopoietin-2:Tie-2 and VEGFR-1:VEGFA. An angiogenesis factor associated with vascular density includes any factor wherein the level of this factor or the ratio of levels of this factor with another factor is statistically shown to correlate with reduced vascular density or increased vascular density. An AGE and/or angiogenesis factor includes CML which is an AGE-product related molecule. Two or more or three or more of CML, VEGFA, VEGFR-1, LMWF, PLGF, HGF, angiopoietin-2:Tie-2 and/or another angiogenesis factor may be determined as well as just one of these factors. Microvascular density includes capillary length density and diffusion radius. Reduced microvascular density is associated with vascular disease. It is proposed herein that at risk (i.e. vulnerable) subjects have reduced vasodilator reserve and increased diffusion radius.

The present disclosure teaches a risk profile to be determined for a subject based on an AGE and/or angiogenesis factor profile and/or level of microvascular density which in turn is suggestive or instructive as to vascular density and in particular, microvascular density. It is proposed that, in relation to heart disease, reduced microvascular density correlates with reduced coronary vasodilator reserve of myocardium. The stratification or profiling enables early diagnosis, confirmation of a clinical diagnosis, treatment monitoring and treatment selection.

Another aspect of the present disclosure provides an assay to identify a subject with vascular disease, the assay comprising determining:

(A) the level or ratio of levels of an AGE and/or angiogenesis factor selected from the list consisting of:
(i) CML;
(ii) VEGFA;
(iii) VEGFR-1;
(iv) Low molecular weight fluorophore (LWF);
(v) Angiopoietin-2;
(vi) Tie-2;
(vii) Endostatin;
(viii) Placental growth factor (PGF); and
(ix) Hepatocyte growth factor (HGF); and/or

(B) the level of microvascular density;

wherein the level or ratio of the AGE and/or angiogenesis factor(s) and/or level of microvascular density relative to a control provides an indication of the presence or absence of vascular disease.

In an embodiment, the AGE and/or angiogenesis profile and/or microvascular density profile is associated with heart disease, the predisposition of development and/or the risk of heart disease and the level of severity and risk of progression. Furthermore, the AGE and/or angiogenesis profile assists in the classification of a subject as being vulnerable or non-vulnerable based on level of vascular density.

Yet another aspect of the present disclosure is directed to an assay to stratify a subject as a vulnerable or non-vulnerable subject with respect to the risk of developing heart disease, the assay comprising determining:

(A) the level or ratio of levels of an AGE and/or angiogenesis factor selected from the list consisting of:
(i) CML;
(ii) VEGFA;
(iii) VEGFR-1;
(iv) Low molecular weight fluorophore (LWF);
(v) Angiopoietin-2;
(vi) Tie-2;
(vii) Endostatin;
(viii) Placental growth factor (PGF); and
(ix) Hepatocyte growth factor (HGF); and/or

(B) the level of microvascular density;

wherein the level or ratio of the AGE and/or angiogenesis factor(s) and/or level of microvascular density relative to a control identifies the subject as being vulnerable or non-vulnerable. In an embodiment, a subject more vulnerable to ischemic insult will exhibit reduced vasodilator reserve and increased diffusion radius which results from a decrease in microvascular density.

Another embodiment enabled herein is an assay to stratify a subject with cardiovascular disease (CAD) with respect to the risk of the subject developing a myocardial infarction (MI), the assay comprising:
(i) selecting a subject having symptoms of CAD or who is at risk of developing CAD; and
(ii) determining the plasma levels of an advanced glycation end (AGE) product or angiogenesis factor selected from the list consisting of carboxymethyl lysine (CML); low molecular weight fluorophore (LMWF); vascular endothelial growth factor-A (VEGF-A); vascular endothelial growth factor receptor-1 (VEGFR-1); angiopoietin-2: Tie-2; endostatin; placental growth factor (PLGF); and hepatocyte growth factor (HGF);
   wherein the subject is stratified as having a MI or is at risk of developing a MI when:
(i) levels of one or more of CML, LMWF, VEGF-A, and/or Tie-2 are reduced compared to a control not having a MI; and/or
(ii) levels of one or more of VEGFR-1, VEGFR-1:VEGFA ratio, angiopoietin-2, angiopoietin-2:Tie-2 ratio, endostatin, PLGF and/or HGF are elevated compared to a control not having a MI.

Still another aspect of the present disclosure contemplates the use of a panel of AGE and/or angiogenesis factors selected from the list consisting of:

(i) CML;

(ii) VEGFA;

(iii) VEGFR-1; and/or

(iv) Low molecular weight fluorophore (LWF);

(v) Angiopoietin-2;

(vi) Tie-2;

(vii) Endostatin;

(viii) Placental growth factor (PGF); and

(ix) Hepatocyte growth factor (HGF); and/or

in the manufacture of an assay to identify the presence, state, classification or progression of vascular disease in a subject.

Yet another aspect taught by the present disclosure is to the use of microvascular density in the manufacture of an assay to identify the presence, state, classification or progression of vascular disease in a subject.

It is proposed herein that subjects with heart disease or who are at risk of developing heart disease have (i) levels of one or more of CML, LMWF, VEGF-A, and/or Tie-2 are reduced compared to a control not having a MI; and/or (ii) levels of one or more of VEGFR-1, VEGFR-1:VEGFA ratio, angiopoietin-2, angiopoietin-2:Tie-2 ratio, endostatin, PLGF and/or HGF are elevated compared to a control not having a MI. Subjects also have a reduced level of microvascular density. A “reduced” level of microvascular density leads to reduced vasodilator reserve and an increased diffusion radius.

Another aspect enabled herein is a method for diagnosing the likelihood of a subject exhibiting reduced coronary microvascular density and thereby at risk of having reduced coronary vasodilator reserve of myocardium remote from the site of an infarction, the method comprising determining:

(A) levels or ratio of levels of CML, VEGFA, VEGFR-1, LMWF, PLGF, HGF, angiopoietin-2:Tie-2 and/or another angiogenesis factor may be determined as well as just one of these factors; and

(B) level of microvascular density;

wherein:
(i) levels of one or more of CML, LMWF, VEGF-A, and/or Tie-2 are reduced compared to a control not having a MI; and/or
(ii) levels of one or more of VEGFR-1, VEGFR-1:VEGFA ratio, angiopoietin-2, angiopoietin-2:Tie-2 ratio, endostatin, PLGF and/or HGF are elevated compared to a control not having a MI.

In an embodiment, the present disclosure teaches a method for diagnosing the likelihood of a subject exhibiting reduced coronary microvascular density and thereby at risk of having reduced coronary vasodilator reserve of myocardium remote from the site of an infarction, the method comprising:
(i) selecting a subject having symptoms of cardiovascular disease (CAD) or who is at risk of developing CAD; and
(ii) determining the levels of an advanced glycation end (AGE) product or angiogenesis factor selected from the list consisting of carboxymethyl lysine (CML); low molecular weight fluorophore (LMWF); vascular endothelial growth factor-A (VEGF-A); vascular endothelial growth factor receptor-1 (VEGFR-1); angiopoietin-2: Tie-2; endostatin; placental growth factor (PLGF) and hepatocyte growth factor (HGF);
   wherein the subject is stratified as having reduced coronary microvascular density or is at risk of developing same when:
(i) levels of one or more of CML, LMWF, VEGF-A, and/or Tie-2 are reduced compared to a control not having a MI; and/or
(ii) levels of one or more of VEGFR-1, VEGFR-1:VEGFA ratio, angiopoietin-2, angiopoietin-2:Tie-2 ratio, endostatin, PLGF and/or HGF are elevated compared to a control not having a MI.

There are many methods which may be used to detect AGE and/or angiogenesis factor levels including mass spectrometry such as liquid chromatography, electrospray ionization-tandem mass spectrometry. Microvascular density may be determined by any number of means and includes measuring capillary length density and diffusion radius.

Immunological assays for the AGE and/or angiogenesis factors can also be done in any convenient format as known in the art. These include Western blots, immunohistochemical assays and ELISA assays. Any means for detecting a level of an AGE and/or angiogenesis factor can be used in accordance with the present disclosure.

The biological sample is any fluid or cell or tissue extract in a subject which comprises the AGE and/or angiogenesis factors. In one embodiment, the biological sample is whole blood or blood plasma. In another embodiment, the biological sample includes serum, lymph, urine, saliva or a cell extract.

The present disclosure teaches the presence of an AGE and/or angiogenesis factor profile and/or a microvascular profile associated with the level of vascular density which is instructive as to the risk of having or developing a vascular disease or condition such as heart disease (e.g. MI) or stroke or other condition. In order to detect an AGE and/or angiogenesis factor, a biological sample is prepared and analyzed for a difference in levels or ratios of levels between the subject being tested and a control. In this context, a “control” includes the levels in a statistically significant comparable population with approximately normal vascular density.

The identification of the association between the pathophysiology of vascular disease and levels of, or ratios of, AGE and/or angiogenesis factors and/or level of microvascular density permits the early presymptomatic screening of individuals to identify those at risk for developing vascular disease or to identify the cause of such a disorder or the risk that any individual will develop same such a cause includes reduced vascular density. The subject assay enables practitioners to identify or stratify individuals who are at risk of developing or progressing with a vascular disease or its manifestations allowing for early intervention. Certain behavioural or therapeutic or dietary protocols may then be introduced to reduce the risk of further developing vascular disease. Presymptomatic diagnosis better enables treatment of vascular disease, including implementing medical therapy. AGE and/or angiogenesis and microvascular density typing of individuals is useful for (a) identifying a form of vascular disease which will respond to particular drugs, (b) identifying types of vascular disease which respond well to specific medications or medication types with fewer adverse effects and (c) guide new drug discovery and testing.

Another aspect taught herein relates to a method of treatment or prophylaxis of a subject comprising assaying the subject with respect to vascular disease by determining:

(A) the level or ratio of levels of an AGE and/or angiogenesis factor selected from the list consisting of:
(i) CML;
(ii) VEGFA;
(iii) VEGFR-1;
(iv) Low molecular weight fluorophore (LWF);
(v) Angiopoietin-2;
(vi) Tie-2;
(vii) Endostatin;
(viii) Placental growth factor (PGF); and
(ix) Hepatocyte growth factor (HGF); and/or

(B) the level of microvascular density;

wherein the level or ratio of the AGE and/or angiogenesis factor(s) and/or microvascular density relative to a control provides a correlation to the presence, state, classification or progression of vascular disease and then providing therapeutic and/or behavioural modification to the subject.

In an embodiment, the present disclosure teaches a method of treatment or prophylaxis of a subject with cardiovascular disease (CAD), the method comprising stratifying the subject with respect to the risk of the subject developing a myocardial infarction (MI), the stratification comprising:
(i) selecting a subject having symptoms of CAD or who is at risk of developing CAD; and
(ii) determining the levels of an advanced glycation end (AGE) product or angiogenesis factor selected from the list consisting of carboxymethyl lysine (CML); low molecular weight fluorophore (LMWF); vascular endothelial growth factor-A (VEGF-A); vascular endothelial growth factor receptor-1 (VEGFR-1); angiopoietin-2:Tie-2; endostatin; placental growth factor (PLGF); and/or hepatocyte growth factor (HGF); and/or
   wherein the subject is stratified as having a MI or is at risk of developing a MI when:
(i) levels of one or more of CML, LMWF, VEGF-A, and/or Tie-2 are reduced compared to a control not having a MI; and/or
(ii) levels of one or more of VEGFR-1, VEGFR-1:VEGFA ratio, angiopoietin-2, angiopoietin-2:Tie-2 ratio, endostatin, PLGF and/or HGF are elevated compared to a control not having a MI; and then providing a therapeutic or behavoral modification to mitigate the risk of the MI.

The present disclosure further teaches a web-based system where data on expression levels of AGE and/or angiogenesis factors are provided by a client server to a central processor which analyzes and compares to a control and optionally considers other information such as patient age, sex, weight and other medical conditions and then provides a report, such as, for example, a risk factor for disease severity or progression or status or an index of probability of heart disease in symptomatic or asymptomatic individuals.

Hence, knowledge-based computer software and hardware are also taught by the present disclosure.

In particular, the assays taught herein may be used in existing or newly developed knowledge-based architecture or platforms associated with pathology services. For example, results from the assays are transmitted via a communications network (e.g. the internet) to a processing system in which an algorithm is stored and used to generate a predicted posterior probability value which translates to the index of disease probability which is then forwarded to an end user in the form of a diagnostic or predictive report.

The assay may, therefore, be in the form of a kit or computer-based system which comprises the reagents necessary to detect the concentration of the AGE and/or angiogenesis biomarkers and/or microvascular density and the computer hardware and/or software to facilitate determination and transmission of reports to a clinician.

Contemplated herein, therefore, is a method of allowing a user to determine the status of a subject with respect to a vascular disease or subtype thereof or stage of vascular disease, the method including:

(a) receiving data in the form of:
(A) levels or concentrations or ratio of levels of an AGE and/or angiogenesis factor selected from the list consisting of:
   (i) CML;
   (ii) VEGFA;
   (iii) VEGFR-1;
   (iv) Low molecular weight fluorophore (LWF);
   (v) Angiopoietin-2;
   (vi) Tie-2;
   (vii) Endostatin;
   (viii) Placental growth factor (PGF); and
   (ix) Hepatocyte growth factor (HGF); and/or
(B) levels of microvascular density;
   wherein the level or ratio of the AGE and/or angiogenesis factor(s) and/or microvascular density relative to a control provides a correlation to the presence, state, classification or progression of vascular disease from the user via a communications network;

(b) processing the subject data via univariate or multivariate analysis to provide a disease index value;

(c) determining the status of the subject in accordance with the results of the disease index value in comparison with predetermined values; and

(d) transferring an indication of the status of the subject to the user via the communications network. Reference to the multivariate analysis includes an algorithm which performs the multivariate or univariate analysis function.

In an embodiment, the present disclosure teaches a method of allowing a user to stratify a subject with cardiovascular disease (CAD) with respect to the risk of developing a myocardial infarction (MI), the method including:
(a) receiving data in the form of levels or concentrations of (i) an advanced glycation end (AGE) product or angiogenesis factor selected from the list consisting of one or more of carboxymethyl lysine (CML); low molecular weight fluorophore (LMWF); vascular endothelial growth factor-A (VEGF-A); vascular endothelial growth factor receptor-1 (VEGFR-1); angiopoietin-2:Tie-2; endostatin; placental growth factor (PLGF); and/or hepatocyte growth factor (HGF);
(b) processing the subject data via univariate or multivariate analysis to provide a risk index value;
(c) determining the status of the subject in accordance with the results of the risk index in comparison with predetermined values; and
(d) transferring an indication of the status of the subject to the user via the communications network;
wherein the status of the subject is that the subject is at risk of having MI or is at risk of developing a MI when:
   (i) levels of one or more of CML, LMWF, VEGF-A, and/or Tie-2 are reduced compared to a control not having a MI; and/or
   (ii) levels of one or more of VEGFR-1, VEGFR-1:VEGFA ratio, angiopoietin-2, angiopoietin-2:Tie-2 ratio, endostatin, PLGF and/or HGF are elevated compared to a control not having a MI.

Conveniently, the method generally further includes:

(a) having the user determine the data using a remote end station; and

(b) transferring the data from the end station to the base station via the communications network.

The base station can include first and second processing systems, in which case the method can include:

(a) transferring the data to the first processing system;

(b) transferring the data to the second processing system; and

(c) causing the first processing system to perform the multivariate analysis function to generate the disease index value.

The method may also include:

(a) transferring the results of the univariate or multivariate analysis function to the first processing system; and

(b) causing the first processing system to determine the status of the subject.

In this case, the method also includes at least one of:

(a) transferring the data between the communications network and the first processing system through a first firewall; and

(b) transferring the data between the first and the second processing systems through a second firewall.

The second processing system may be coupled to a database adapted to store predetermined data and/or the univariate or multivariate analysis function, the method includes:

(a) querying the database to obtain at least selected predetermined data or access to the univariate or multivariate analysis function from the database; and

(b) comparing the selected predetermined data to the subject data or generating a predicted probability index.

The second processing system can be coupled to a database, the method including storing the data in the database.

The method can also include having the user determine the data using a secure array, the secure array of elements capable of determining the level of angiogenesis factor(s) and having a number of features each located at respective position(s) on the respective code. In this case, the method typically includes causing the base station to:

(a) determine the code from the data;

(b) determine a layout indicating the position of each feature on the array; and

(c) determine the parameter values in accordance with the determined layout, and the data.

The method can also include causing the base station to:

(a) determine payment information, the payment information representing the provision of payment by the user; and

(b) perform the comparison in response to the determination of the payment information.

The present disclosure teaches a base station for determining the status of a subject with respect to a vascular disease or a subtype thereof or a stage of vascular disease, the base station including:

(a) a storage method;

(b) a processing system, the processing system being adapted to:

(c) receive subject data from a user via a communications network, the data including:
(A) levels or concentrations of an AGE and/or angiogenesis factor selected from the list consisting of:
   (i) CML;
   (ii) VEGFA;
   (iii) VEGFR-1;
   (iv) Low molecular weight fluorophore (LWF);
   (v) Angiopoietin-2;
   (vi) Tie-2;
   (vii) Endostatin;
   (viii) Placental growth factor (PGF); and
   (ix) Hepatocyte growth factor (HGF); and/or
(B) levels of microvascular density;
   wherein the level or ratio of the AGE and/or angiogenesis factor(s) and/or microvascular density relative to a control provides a correlation to the presence, state, classification or progression of vascular disease;

(d) performing an algorithmic function including comparing the data to predetermined data;

(e) determining the status of the subject in accordance with the results of the algorithmic function including the comparison; and

(c) output an indication of the status of the subject to the user via the communications network.

The processing system can be adapted to receive data from a remote end station adapted to determine the data.

The processing system may include:

(a) a first processing system adapted to:
(i) receive the data; and
(ii) determine the status of the subject in accordance with the results of the univariate or multivariate analysis function including comparing the data; and

(b) a second processing system adapted to:
(i) receive the data from the processing system;
(ii) perform the univariate or multivariate analysis function including the comparison; and
(iii) transfer the results to the first processing system.

The base station typically includes:

(a) a first firewall for coupling the first processing system to the communications network; and

(b) a second firewall for coupling the first and the second processing systems.

The processing system can be coupled to a database, the processing system being adapted to store the data in the database.

In another embodiment, contemplated herein is an assay for determining the presence of heart disease in a subject, the assay comprising determining:

(A) the concentration of an AGE and/or angiogenesis factor selected from the list consisting of:
(i) CML;
(ii) VEGFA;
(iii) VEGFR-1;
(iv) Low molecular weight fluorophore (LWF);
(v) Angiopoietin-2;
(vi) Tie-2;
(vii) Endostatin;
(viii) Placental growth factor (PGF); and
(ix) Hepatocyte growth factor (HGF); and/or

(B) level of microvascular density;

wherein the level or ratio of the AGE and/or angiogenesis factor(s) and/or microvascular density relative to a control provides a correlation to the presence, state, classification or progression of heart disease in a biological sample from the subject wherein an altered concentration in the angiogenesis factor is indicative of the subject having heart disease.

By “heart disease” includes MI or other conditions as listed above.

In accordance with this embodiment, levels of the AGE and/or angiogenesis factor and/or microvascular density may be screened alone or in combination with other biomarkers or heart or vascular disease indicators. An “altered” level means an increase or elevation or a decrease or reduction in the concentrations of the AGE and/or angiogenesis factors. It is proposed herein that the level or ratio of AGE and/or angiogenesis factors and/or level of microvascular density are indicative of vascular density.

The determination of the concentrations or levels of the AGE and/or angiogenesis factors and determining the level of microvascular density enable establishment of a diagnostic rule based on the concentrations relative to controls. Alternatively, the diagnostic rule is based on the application of a statistical and machine learning algorithm. Such an algorithm uses relationships between AGE and/or angiogenesis factors and disease status observed in training data (with known disease status) to infer relationships which are then used to predict the status of patients with unknown status. An algorithm is employed which provides an index of probability that a patient has heart disease or a state or form or class thereof. The algorithm performs a univariate or multivariate analysis function.

Hence, the present disclosure teaches a diagnostic rule based on the application of statistical and machine learning algorithms. Such an algorithm uses the relationships between AGE and/or angiogenesis factors and/or microvascular density and disease status observed in training data (with known disease status) to infer relationships which are then used to predict the status of patients with unknown status. Practitioners skilled in the art of data analysis recognize that many different forms of inferring relationships in the training data may be used without materially changing the present disclosure.

Hence, the present disclosure contemplates the use of a knowledge base of training data comprising levels of AGE and/or angiogenesis factors and/or level of microvascular density from a subject with a vascular condition to generate an algorithm which, upon input of a second knowledge base of data comprising levels of the same angiogenesis factors and/or level of microvascular density from a patient with an unknown vascular disease condition, provides an index of probability that predicts the nature of the vascular disease condition.

The term “training data” includes knowledge of levels of AGE and/or angiogenesis factors relative to a control. A “control” includes a comparison to levels of AGE and/or angiogenesis factors in a subject devoid of the vascular disease condition or cured of the condition or may be a statistically determined level based on trials. The term “levels” also encompasses ratios of levels of AGE and/or angiogenesis factors.

Hence, the “training data” includes levels or ratios of one or more AGE and/or angiogenesis factors selected from

(i) CML;

(ii) VEGFA;

(iii) VEGFR-1;

(iv) Low molecular weight fluorophore (LWF);

(v) Angiopoietin-2;

(vi) Tie-2;

(vii) Endostatin;

(viii) Placental growth factor (PGF);

(ix) Hepatocyte growth factor (HGF); and/or

(x) level of microvascular density.

The levels or concentrations of the AGE and/or angiogenesis factors and/or level of microvascular density provide the input test data referred to herein as a “second knowledge base of data”. The second knowledge base of data either is considered relative to a control or is fed into an algorithm generated by a “first knowledge base of data” which comprise information of the levels of angiogenesis factors in a subject with a known vascular disease condition. The second knowledge base of data is from a subject of unknown status with respect to a vascular disease condition. The output of the algorithm or the comparison to a control is a probability or risk factor, referred to herein as “an index of probability”, of a subject having a particular heart disease condition or not having the condition. This includes determining whether the subject has unstable (vulnerable patient) or stable (non-vulnerable patient) plaques.

Data generated from the levels of an AGE and/or angiogenesis factor selected from the list consisting of:

(i) CML;

(ii) VEGFA;

(iii) VEGFR-1;

(iv) Low molecular weight fluorophore (LWF);

(v) Angiopoietin-2;

(vi) Tie-2;

(vii) Endostatin;

(viii) Placental growth factor (PGF);

(ix) Hepatocyte growth factor (HGF); and/or

(x) level of microvascular density;

are input data. The input of data comprising the AGE and/or angiogenesis factors and/or level of microvascular density is/are compared with a control or is put into the algorithm which provides a risk value of the likelihood that the subject has, for example, vascular disease such as heart disease. A treatment regime can also be monitored as well as a likelihood of a relapse.

In context of the present disclosure, “fluid” includes any blood fraction, for example serum or plasma, that can be analyzed according to the methods described herein. By measuring blood levels of a particular AGE and/or angiogenesis factors, it is meant that any appropriate blood fraction can be tested to determine blood levels and that data can be reported as a value present in that fraction. Other fluids contemplated herein include saliva, cell extracts, ascites, tissue exudate, urine, lymph fluid, mucus and respiratory fluid.

As described above, methods for diagnosing vascular disease by determining levels of specific identified AGE and/or angiogenesis factors and using these levels as second knowledge base data in an algorithm generated with first knowledge base data or levels of the same biomarkers in patents with a known disease. Also provided are methods of detecting symptomatic vascular disease comprising determining the presence and/or velocity of specific identified angiogenesis factors in a subject's sample. By “velocity” it is meant the change in the concentration of the AGE and/or angiogenesis factor in a patient's sample over time. Microvascular density is also considered a biomarker.

The term “sample” as used herein means any sample containing lipid analytes that one wishes to detect including, but not limited to, biological fluids (including blood, plasma, serum, ascites), saliva, tissue extracts, freshly harvested cells, and lysates of cells which have been incubated in cell cultures. In a particular embodiment, the sample is heart tissue, blood, serum, plasma or ascites.

As indicated above, the “subject” can be any mammal, generally human, suspected of having or having vascular disease. The subject may be symptomatic or asymptomatic.

The term “control sample” includes any sample that can be used to establish a first knowledge base of data from subjects with a known disease status.

The method of the subject disclosure may be used in the diagnosis and staging of vascular disease. The present disclosure also teaches the monitoring of the progression of a condition and to monitor whether a particular treatment is effective or not. In particular, the method can be used to confirm the absence or amelioration of the symptoms of the condition such as following surgery, stents, medication or behavioural change.

In an embodiment, the subject disclosure contemplates a method for monitoring the progression of vascular disease in a patient, comprising:

(a) providing a sample from a patient;

(b) determining the level of an AGE and/or angiogenesis factor selected from the list consisting of:
(i) CML;
(ii) VEGFA;
(iii) VEGFR-1;
(iv) Low molecular weight fluorophore (LWF);
(v) Angiopoietin-2;
(vi) Tie-2;
(vii) Endostatin;
(viii) Placental growth factor (PGF);
(ix) Hepatocyte growth factor (HGF); and/or
(x) determining the level of microvascular density;
   wherein the level or ratio of the AGE and/or angiogenesis factor(s) and/or level of microvascular density relative to a control provides a correlation to the presence, state, classification or progression of vascular disease subjecting the levels to an algorithm to provide an index of probability of the patient having heart disease; and

(c) repeating steps (a) and (b) at a later point in time and comparing the result of step (b) with the result of step (c) wherein a difference in the index of probability is indicative of the progression of the condition in the patient.

In particular, an increased index of probability of a disease condition at the later time point may indicate that the condition is progressing and that the treatment (if applicable) is not being effective. In contrast, a decreased index of probability at the later time point may indicate that the condition is regressing and that the treatment (if applicable) is effective.

Reference to an “algorithm” or “algorithmic functions” as outlined above includes the performance of a univariate or multivariate analysis function. A range of different architectures and platforms may be implemented in addition to those described above. It will be appreciated that any form of architecture suitable for implementing the present disclosure may be used. However, one beneficial technique is the use of distributed architectures. In particular, a number of end stations may be provided at respective geographical locations. This can increase the efficiency of the system by reducing data bandwidth costs and requirements, as well as ensuring that if one base station becomes congested or a fault occurs, other end stations could take over. This also allows load sharing or the like, to ensure access to the system is available at all times.

In this case, it would be necessary to ensure that the base station contains the same information and signature such that different end stations can be used.

It will also be appreciated that in one example, the end stations can be hand-held devices, such as PDAs, mobile phones, or the like, which are capable of transferring the subject data to the base station via a communications network such as the Internet, and receiving the reports.

In the above aspects, the term “data” means the levels or concentrations of the angiogenesis factors. The “communications network” includes the internet. When a server is used, it is generally a client server or more particularly a simple object application protocol (SOAP).

A report outlining the likelihood of vascular disease by the subject is issued.

In accordance with the present disclosure myocardium with reduced vascular density is more likely to suffer ischemic damage. Furthermore, reduced vascular density is proposed herein to contribute mechanistically to ischemic damage. Without intending to limit the present disclosure to any one theory or mode of action, it is proposed that myocardium with reduced vascular density is less able to survive or escape damage from ischemic insult. This vulnerability to ischemic insult is due in part to reduced vasodilator reserve and increased diffusion radius.

The teachings and methods herein do not require the determine of microvascular density. A determination need only be made of the biochemical markers.

Aspects contemplated herein are further described by the following non-limiting Examples. Materials and Methods used in these Examples are provided below.

### Methods

### Study Population

Patients scheduled for cardiac surgery were recruited to the a cardiac tissue bank.

Patients recruited to the tissue bank were unselected apart from the exclusion of patients with previous cardiac surgery or who were at particularly high surgical risk. All blood samples were collected from the radial artery cannula of fasted patients before anaesthesia and plasma stored at −80° C. A 3-mm partial-thickness biopsy was taken during surgery, immediately after cardioplegia, from a region of the lateral wall of the left ventricle near the base of the heart, between the territories of the left anterior descending and circumflex arteries, that was free of any macroscopic pathology. The biopsy was immediately rinsed in ice-cold normal saline with 20 mmol/L KC1 to ensure cardiomyocytes and vessels were relaxed. Part of the biopsy was fixed in 4% v/v paraformaldehyde and embedded in paraffin, and part was frozen in Optimum Cutting Temperature compound (Tissue-Tek, Sakura Finetek Europe B. V., Alphen aan den Rijn, The Netherlands) for frozen section. Clinical and surgical details were recorded for each patient. All patients had Swan-Ganz catheters inserted before surgery that provided measures of pulmonary artery and pulmonary capillary wedge pressures and cardiac output recorded immediately after induction of anaesthesia.

A total of 84 patients were selected from the tissue bank who had coronary artery bypass graft surgery alone and who did not have heart failure or atrial fibrillation, had not received thrombolysis or furosemide therapy, and did not have evidence of previous ST-segment-elevation myocardial infarction, such as pathological Q waves on electrocardiography, akinesis or dyskinesis on ventriculogram or echocardiography, or scars visible on inspection of the heart at surgery. Fifty-seven patients had no evidence of previous myocardial infarction and 27 patients had previous documented non-ST-segment-elevation myocardial infarction (NSTEMI). Although most no myocardial infarction (no-MI) patients had preceding angina, six patients without angina came to coronary artery surgery after medical workup for planned major non-cardiac surgery or had a strong family history of coronary artery disease. All patients with NSTEMI presented with symptoms of myocardial ischemia associated with elevation of serum troponin I level according to the local hospital's standard. All patients had normal or near-normal left ventricular systolic function as assessed by pre-operative transthoracic echocardiography and/or ventriculogram, with left ventricular ejection fraction ≧50%.

### Biochemistry

Blood hemoglobin and HbA 1 c, and plasma levels of glucose, insulin, lipids, and creatinine were measured by a pathology service using routine clinical methods. Amino-terminal-pro-B-type natriuretic peptide (NT-proBNP) was measured by electrochemiluminescence immunoassay using an Elecsys instrument (Roche Diagnostics, Basel, Switzerland). CML, VEGFA and VEGFR-1 were determined in blood plasma using standard clinical methods.

### Histological Analysis

All histological analyses were performed blind to patient group allocation. Picrosirius red-stained 4 μm paraffin sections were analyzed for interstitial and perivascular fibrosis and arteriolar dimensions by quantitative morphometry of digitized images of the whole section (Aperio Technologies, Inc., CA). Myocardial interstitial collagen density was calculated using the positive pixel count algorithm as the area of collagen staining expressed as a percentage of the total myocardial tissue area, after excluding perivascular fibrosis and the pericardium.

Arterioles were identified by the presence of a layer of media and immunohistochemical staining for elastin showed the blood vessels were relaxed. The tissue was not perfusion fixed and the arterioles were usually oval in shape because of deformation and/or because they were cut at an oblique angle. Arterioles counted for estimation of arteriolar density and analyzed for perivascular fibrosis had diameters (average of maximum and minimum diameter of each arteriole) of 12-151 μm. Perivascular fibrosis was calculated as the ratio of the area of perivascular fibrosis to the total vessel area (area of vessel wall plus lumen) as determined by planimetry (Tomita et al., Hypertension 32:273-279, 1998). Arteriolar wall area and circumference were measured for arterioles with average diameters of 20-80 μm.

Cardiomyocyte width, determined on 4 μm sections of paraffin-embedded tissue stained for reticulin (Gordon and Sweets Am J Pathol 12:545-552, 1936) was the mean of >100 measurements for each section of the shortest diameter of cardiomyocyte profiles containing a nucleus. Cardiomyocyte triglyceride was determined by Oil Red O stain of frozen sections and analyzed as the area of triglyceride staining expressed as a percentage of the total myocardial tissue area, after excluding the pericardium and any adherent tissue.

Capillary length density, which is the length of capillaries per unit volume of tissue, was determined by analysis of 4 μm sections of paraffin-embedded tissue immunostained for CD31 (mouse anti-human CD31 monoclonal antibody, Dako Denmark A/S, Glostrup, Denmark) using standard stereological techniques (Lim et al., Pediatr Res 60:83-87, 2006). Each tissue section was systematically imaged over the entire tissue face using 40× objective (SPOT Insight 4 Meg FW Color Mosaic camera, model 14.2, Diagnostic Instruments, Inc., MI). Each image was opened in Image-Pro Plus image analysis software (SciTech, Australia) and a grid (40 μm×40 μm) overlaid onto the image. Four unbiased counting frames (9×9 squares) were used, one in each quarter of the entire grid. There were two lines of exclusion for each counting frame, one horizontal running along the bottom of the grid and extending down one grid square, and the other running vertical along the left border of the counting frame, extending up one grid square. For each counting frame, the following was recorded:

Pt=the number of grid points lying on tissue (maximum of 9).

C=the number of capillaries found within the counting frame, excluding those that touched a line of exclusion.

Capillary length density was calculated using the equation:

Length density=2·ΣC÷ΣPt·0.0016 mm², where 0.0016 mm²=area of each grid square (Black et al., J Hypertens 19:785-794, 2001).

Diffusion radius=√(1±7C×length density) [Lim et al., Supra 20016]

### Statistical Analysis

The significance of differences between the two study groups was determined by analysis of variance for continuous variables and Fisher's exact test for categorical variables. Continuous data were logarithmically transformed when necessary to normalize variances. The Fisher's Protected Least Significant Difference test was used for multiple comparisons. Calculations were done with Statview 5.0.1 statistical software (SAS Institute Inc.) and a P value of less than 0.05 was considered to indicate statistical significance.

### Example 1

### Study Patients

The patient characteristics are shown in Table 1. The median duration of angina before surgery for the no-MI patients was 6 (range 0.75-360, N=51) months and an additional six patients did not have angina before surgery. The median duration of angina before NSTEMI was 2 (range 0.1-60, N=19) months, and an additional eight patients did not experience angina before their NSTEMI. The median time between NSTEMI and surgery was 12 (range 5-89) days and the median maximum troponin I was 1.2 (0.16-33.5) μg/L for NSTEMI patients. The site of NSTEMI, based on ECG and ventricular wall hypokinesis on ventriculogram and/or echocardiogram was anterior in eight patients, inferior in four patients, antero-lateral in one patient, and indeterminate in 14 patients. Of the 27 NSTEMI patients, 12 experienced angina after myocardial infarction and 15 had no further episodes of chest pain before surgery. The two patient groups were of similar age, had similar extent of coronary artery disease, occluded coronary arteries, collaterals, and wall motion abnormalities, and had similar clinical and biochemical characteristics, apart from lower total and low density lipoprotein cholesterol, and 2.5-fold higher plasma NT-proBNP level in NSTEMI patients. There were no differences in therapies, except that no-MI patients were more likely to have ceased aspirin therapy 1-2 weeks before surgery (33% vs. 11%, P=0.04) and NSTEMI patients were more likely to have received heparin or enoxaparin before surgery (33% vs. 11%, P=0.02).

Among the patients who had pre-operative transthoracic echocardiography, the two patient groups had similar left ventricular ejection fraction (no-MI:61±1%, mean±SEM, N=37; NSTEMI:62±2%, N=20), left ventricular end-diastolic diameter (no-MI:4.9±0.1 cm, N=24; NSTEMI:4.9±0.2 cm, N=13), mitral Doppler flow velocity E/A wave ratio (no-MI:1.0±0.1, N=29; NSTEMI:0.9±0.1, N=15), mitral valve deceleration time (no-MI:228±8 msec, N=33; NSTEMI:249±19 msec, N=16), early diastolic peak velocity of the septal mitral annulus, E′ (no-MI:6.0±0.3 cm/sec, N=28; NSTEMI:5.7±0.4 cm/sec, N=12), and E/E′ ratio (no-MI:12.2±0.7, N=31; NSTEMI:11.5±1.0, N=13).

### Example 2

### Histology

The histology of the left ventricular biopsies is shown in Table 2. There were no differences between no-MI and NSTEMI patients in interstitial or perivascular fibrosis, arteriolar dimensions, or in cardiomyocyte width, although the cardiomyocyte width/body surface area ratio was slightly but significantly increased in NSTEMI patients. In comparison with no-MI patients, NSTEMI patients had 47% lower arteriolar density, 40% lower capillary length density, and 38% less cardiomyocyte triglyceride. The 32% greater diffusion radius in NSTEMI patients was independent of cardiomyocyte size, as shown by the 28% higher diffusion radius/cardiomyocyte width ratio.

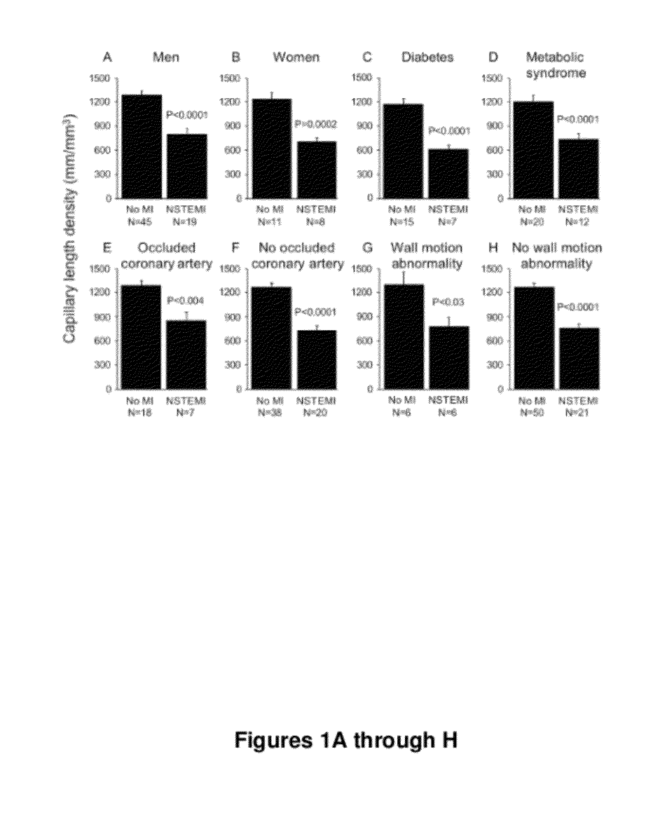

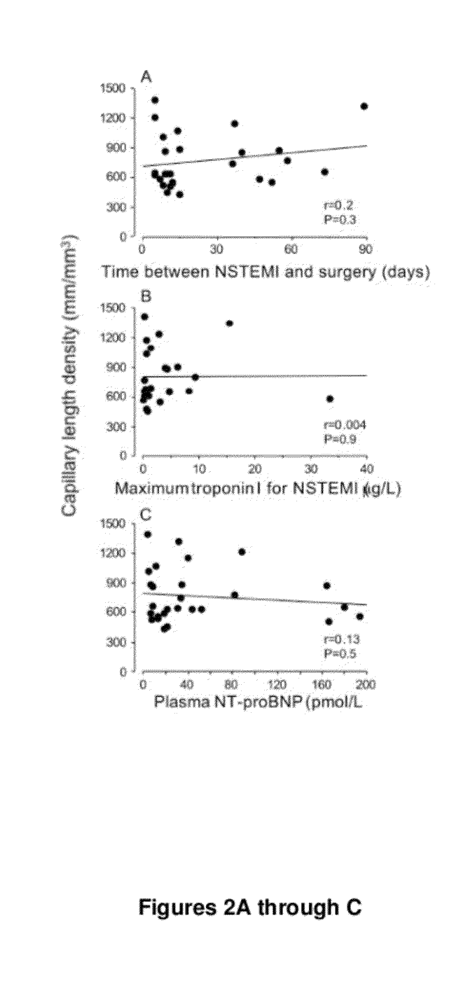

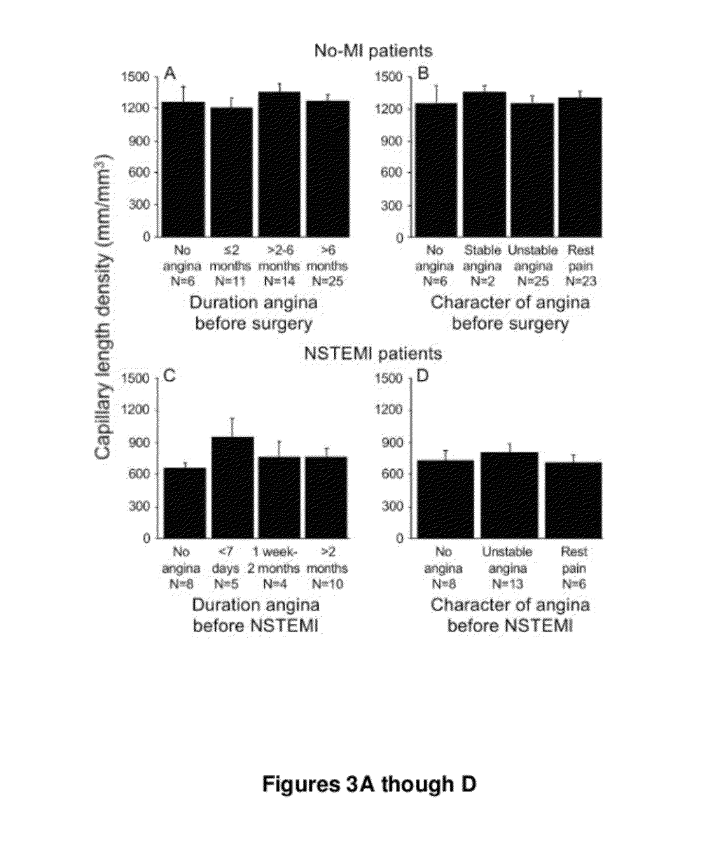

The reduced capillary length density of NSTEMI patients was similar for both men and women, for patients with diabetes or metabolic syndrome, and for patients with or without occluded coronary arteries or wall motion abnormalities (FIG. 1). There was no significant correlation between capillary length density and either time between myocardial infarction and surgery, maximum plasma troponin I level, or plasma NT-proBNP level in NSTEMI patients (FIG. 2). Moreover, capillary length density was not related to either the duration or character of angina before surgery for no-MI patients, or before myocardial infarction for the NSTEMI patients (FIG. 3). The same relationships were found for arteriolar density as for capillary length density.

### Example 3

### Determination of Angiogenesis Factors

The angiogenesis factors CML, VEGFA and VEGFR-1 were determined Results are provided below as means±SEM.

NSTEMI patients had reduced plasma carboxymethyl lysine (μmol/L): No-MI:2.13±0.07, NSTEMI:1.40±0.13, P<0.0001.

NSTEMI patients had reduced plasma levels of VEGF-A (pg/ml): No-MI:26.4±2.2, NSTEMI:19.6±3.0, P=0.030, P=0.083.

NSTEMI patients had increased plasma levels of VEGF receptor-1 (pg/ml): No-MI:139±14, NSTEMI:251±44, P=0.002.

NSTEMI patients had increased VEGF-R1/VEGF-A ratio (pg/pg): No-MI:13.5±5, NSTEMI:18.8±4.7, P=0.025.

These data indicate that plasma levels of CML, VEGF-A, and VEGF-R1 can aid the identification of individuals with impaired myocardial angiogenesis who are at increased risk of MI.

Characteristics of patients undergoing coronary artery bypass surgery without previous myocardial infarction (no-MI) or with recent non-ST-segment-elevation myocardial infarction (NSTEMI) are shown in Table 1.

TABLE 1

| | No-MI | NSTEMI | P |
|---|---|---|---|
| Characteristic | (N = 57) | (N = 27) | Value |
| Age, years | 64 ± 1 | 66 ± 2 | NS |
| Male sex, n (%) | 46 (81%) | 19 (70%) | NS |
| Left main stenosis >50%, n (%) | 21 (37%) | 9 (33%) | NS |
| One vessel stenosis >70%, n (%) | 11 (19%) | 8 (30%) | NS |
| Two vessel stenosis >70%, n (%) | 21 (37%) | 9 (33%) | NS |
| Three vessel stenosis >70%, n (%) | 21 (37%) | 10 (37%) | NS |
| Patients with occluded coronary | 19 (33%) | 7 (26%) | NS |
| artery, n (%) | | | |
| Coronary collaterals, Rentrop | 21 (37%) | 9 (33%) | NS |
| grade 2 or 3, n (%) | | | |
| Wall motion abnormality | 6 (11%) | 6 (22%) | NS |
| Previous percutaneous transluminal | 7 (12%) | 0 (0%) | NS |
| coronary angioplasty, n (%) | | | |
| CABG conduits/patient, n | 3.4 ± 0.1 | 3.7 ± 0.2 | NS |
| Body mass index (kg/m²) | 29.3 ± 0.7 | 29.0 ± 0.8 | NS |
| Body surface area (m²) | 1.98 ± 0.02 | 1.90 ± 0.04 | NS |
| Clinical risk factors | | | |
| Diabetes, n (%) | 15 (26%) | 7 (26%) | NS |
| Metabolic syndrome (non- | 21 (37%) | 12 (44%) | NS |
| diabetic), n (%) | | | |
| Pre-admission SBP (mmHg) | 133 + 2 | 133 + 3 | NS |
| Pre-admission DBP (mmHg) | 75 + 1 | 77 + 2 | NS |
| Hypertension, n (%) | 39 (68%) | 20 (74%) | NS |
| Use of tobacco, ever, n (%) | 33 (58%) | 18 (67%) | NS |
| Fasting plasma total | 3.5 ± 0.1 | 3.0 ± 0.1 | 0.02 |
| cholesterol (mmol/L) | | | |
| Fasting plasma LDL | 2.1 ± 0.1 | 1.6 ± 0.1 | 0.02 |
| cholesterol (mmol/L) | | | |
| Fasting plasma HDL | 0.96 ± 0.03 | 0.91 ± 0.07 | NS |
| cholesterol (mmol/L) | | | |
| Fasting plasma | 1.7 ± 0.1 | 1.4 ± 0.1 | NS |
| triglyceride (mmol/L) | | | |
| Fasting plasma glucose (mmol/L) | 6.2 ± 0.2 | 6.3 ± 0.2 | NS |
| Fasting plasma insulin (pmol/L) | 83 ± 11 | 63 ± 8 | NS |
| β cell function from HOMA2-% B | 82 ± 6 | 66 ± 5 | NS |
| Insulin sensitivity from | 105 ± 9 | 115 ± 13 | NS |
| HOMA2-% S | | | |
| Insulin resistance from | 1.50 ± 0.16 | 1.23 ± 0.16 | NS |
| HOMA2-IR | | | |
| Plasma NT-proBNP (pmol/L) | 20 ± 4 | 49 ± 11 | 0.001 |
| Hemoglobin (g/L) | 14.0 ± 0.2 | 13.7 ± 0.3 | NS |
| Plasma creatinine (μmol/L) | 92 ± 2 | 101 ± 7 | NS |
| eGFR (mL/min per 1.73 m²) | 71 ± 2 | 65 ± 3 | NS |
| C-reactive protein (mg/L) | 4.7 ± 1.1 | 6.2 ± 1.5 | NS |
| Medications | | | |
| ACE inhibitor therapy, n (%) | 31 (54%) | 18 (67%) | NS |
| ARB therapy, n (%) | 15 (26%) | 6 (22%) | NS |
| ACEI and/or ARB therapy, (%) | 44 (77%) | 22 (81%) | NS |
| Statin therapy, n (%) | 48 (84%) | 26 (96%) | NS |
| Aspirin therapy, n (%) | 52 (91%) | 26 (96%) | NS |
| Calcium antagonist | 15 (26%) | 9 (33%) | NS |
| therapy, n (%) | | | |
| β-blocker therapy, n (%) | 43 (75%) | 21 (78%) | NS |
| Long-acting nitrate | 14 (25%) | 12 (44%) | NS |
| therapy, n (%) | | | |
| Thiazide or indapamide | 15 (26%) | 4 (15%) | NS |
| therapy, n (%) | | | |
| Intra-operative hemodynamics | | | |
| immediately post induction | | | |
| of anesthesia | | | |
| Central venous pressure (mmHg) | 8.6 ± 0.5 | 7.5 ± 0.7 | NS |
| Pulmonary capillary wedge | 10.4 ± 0.5 | 10.5 ± 0.4 | NS |
| pressure (mmHg) | | | |
| Cardiac index (litres/min/m²) | 2.5 ± 0.1 | 2.6 ± 0.1 | NS |

Data shown as means±SEM or n (%). Coronary collaterals were scored according to Rentrop et al., J Am Coll Cardiol 5:587-592, 1985. Metabolic syndrome was defined according to the Adult treatment Panel III guidelines (Grundy et al., Circulation 112:2735-2752, 2005). ACE, angiotensin converting enzyme; ARB, angiotensin receptor blocker; eGFR, estimated glomerular filtration rate calculated using the Modification of Diet in Renal Disease study equation; (Levey et al., Ann Intern Med 130:461-470, 1999) HDL, high density lipoprotein; HOMA, Homeostasis Model Assessment calculator version 2.2; (Wallace et al., Diabetes Care 27:1487-1495, 2004) LDL, low density lipoprotein.

The histology of left ventricular biopsies of patients undergoing coronary artery bypass surgery without previous myocardial infarction (no-MI) or with recent non-ST-segment-elevation myocardial infarction (NSTEMI) are shown in Table 2.

TABLE 2

| | No-MI | NSTEMI | P |
|---|---|---|---|
| Characteristic | (N = 57) | (N = 27) | Value |
| Myocardium area per | 4.2 ± 0.3 | 5.6 ± 0.8 | 0.04 |
| section (mm²) | | | |
| Interstitial fibrosis (%) | 1.4 ± 0.1 | 1.4 ± 0.1 | NS |
| Perivascular fibrosis ratio | 1.9 ± 0.1 | 1.5 ± 0.2 | NS |
| Cardiomyocyte width (μm) | 22.6 ± 0.5 | 23.4 ± 0.6 | NS |
| Cardiomyocyte width/body | 11.5 ± 0.2 | 12.4 ± 0.4 | 0.02 |
| surface area ratio (μm/m²) | | | |
| Number of arterioles per | 3.9 ± 0.2 | 2.6 ± 0.4 | 0.003 |
| section | | | |
| Arterioles/mm² myocardium | 1.10 ± 0.08 | 0.58 ± 0.10 | 0.0001 |
| area | | | |
| Mean arteriolar | 40 ± 2 | 40 ± 3 | NS |
| diameter (μm) | | | |
| Arteriolar wall area/ | 5.3 ± 0.2 | 5.4 ± 0.4 | NS |
| circumference ratio | | | |
| (μm²/μm) | | | |
| Capillary length density | 1279 ± 42 | 768 ± 52 | <0.0001 |
| (mm/mm³) | | | |
| Diffusion radius (μm) | 16.1 ± 0.3 | 21.2 ± 0.6 | <0.0001 |
| Diffusion radius/body | 8.2 ± 0.1 | 11.2 ± 0.4 | <0.0001 |
| surface area ratio | | | |
| (μm/m²) | | | |
| Diffusion radius/ | 0.72 ± 0.01 | 0.92 ± 0.03 | <0.0001 |
| cardiomyocyte width | | | |
| ratio (μm/μm) | | | |
| Cardiomyocyte | 3.2 ± 0.3 | 2.0 ± 0.2 | 0.01 |
| triglyceride: Oil Red | | | |
| 0 stain (% area) | | | |

Data shown as means±SEM. Myocardium area per section excludes pericardium and adherent connective tissue. No arterioles were identified in sections from four NSTEMI patients; thus, perivascular fibrosis was measured for 23 NSTEMI patients. Arteriolar wall area/circumference ratio was measured for arterioles with diameter (average of maximum and minimum diameter of each arteriole) of 20-80 μm for 56 no-MI and 21 NSTEMI patients. Capillary length density and diffusion radius were measured for 56 no-MI patients and cardiomyocyte triglyceride was measured for 26 NSTEMI patients. Whereas the analysis of capillary length density included all capillaries, both in cross-section and longitudinal, only arterioles in approximate cross-section with diameters 12-151 μm were included in the estimate of the number of arterioles per section.

### Example 4

### Reduced Microvascular Density in Non-Ischemic Myocardium of Patients with Recent Non-ST-Segment Myocardial Infarction

Myocardial infarction is associated with reduced coronary vasodilator reserve and maximal blood flow not only in the infarcted myocardium but also in myocardium remote from the site of infarction.

A hypothesis was tested that patients with myocardial infarction have lower microvasculature density in myocardium remote from the site of infarction than patients with similar extent of coronary artery disease (CAD) without myocardial infarction and examined the relationship between myocardial capillary length density and plasma levels of advanced glycation endproducts (AGEs) and angiogenesis markers.

Biopsies were analyzed from non-ischemic left ventricular (LV) myocardium and measured plasma levels of AGEs and angiogenesis markers in patients undergoing coronary artery bypass graft surgery, 57 without previous myocardial infarction (no MI) and 27 with recent non-ST-segment-elevation myocardial infarction (NSTEMI). Comparison was made with biopsies from 31 aortic stenosis (AS) patients and 6 patients with “normal” LV without CAD.

NSTEMI patients and AS patients had similar arteriolar and capillary length densities, which were approximately half the densities of no-MI patients (p<0.0001), and NSTEMI patients had a higher diffusion radius/cardiomyocyte width ratio than no-MI, “normal” LV and AS patients, NSTEMI patients also had lower plasma levels of carboxymethyl lysine (p<0.0001) and low molecular weight fluorophore (LMWF, p-0.028), and increased vascular endothelial growth factor (VEGF) receptor-1/VEGF-A ratio (p-0.014), and endostatin (p=0.03) and hepatocyte growth factor levels (p=0.0005) than no-MI patients. Moreover, LMWF (p=0.019) and VEGF receptor-1 levels (p=0.049) correlated with myocardial capillary length density in patients without myocardial infarction. The results are shown in Table 3.

TABLE 3
Plasma levels of advanced glycation endproducts and
angiogenesis-related biomarkers in patients undergoing
coronary artery bypass graft surgery without previous
myocardial infarction (no-MI) or with recent non-ST-
segment-elevation myocardial infarction (NSTEMI).

| | No-MI | | NSTEMI | | p |
|---|---|---|---|---|---|
| Parameter | (n = 57) | | (n = 26) | | Value |
| CML (μmol/L) | 2.2 | (1.8, 2.4) | 1.4 | (0.9, 1.7) | <0.0001 |
| LMWF (AU/mL) | 2.5 | (2.1, 3.2) | 1.9 | (1.2, 3.1) | 0.028 |
| sRAGE | 612 | (456, 814) | 583 | (417, 719) | 0.40 |
| VEGF-A (pg/mL) | 24 | (13, 34) | 19 | (12, 23) | 0.057 |
| VEGF-B (pg/mL) | <15 | | <15 | | |
| VEGFR-1 (pg/mL) | 104 | (72, 161) | 185 | (80, 329) | 0.056 |
| VEGFR-1/VEGF-A | 4.3 | (2.6, 9.8) | 10.6 | (4.0, 23.7) | 0.014 |
| ratio (pg/pg) | | | | | |
| VEGFR-2 (pg/mL) | 6637 | (5965, 7594) | 6882 | (6034, 7394) | 0.67 |
| Angiopoietin-1 | 3666 | (2405, 5730) | 4470 | (1914, 5496) | 0.93 |
| (pg/mL) | | | | | |
| Angiopoietin-2 | 1366 | (1110, 1972) | 1504 | (1314, 2100) | 0.15 |
| (pg/mL) | | | | | |
| Tie-1 (ng/mL) | 36 | (29, 41) | 33 | (28, 42) | 0.55 |
| Tie-2 (ng/mL) | 15.5 | (13.2, 17.5) | 13.2 | (11.7, 16.5) | 0.059 |
| Angiopoietin-2/ | 92 | (70, 129) | 122 | (92, 163) | 0.017 |
| Tie-2 ratio (pg/ng) | | | | | |
| FGF acidic (pg/mL) | <30 | | <30 | | |
| FGF basic (pg/mL) | 8.3 | (4.8, 10.4) | 8.6 | (4.7, 9.3) | 0.89 |
| Endostatin (ng/mL) | 93 | (75, 115) | 111 | (79, 159) | 0.030 |
| PLGF (pg/mL) | 10.8 | (8.0, 15.1) | 12.3 | (9.6, 15.7) | 0.11 |
| HGF (pg/mL) | 720 | (576, 961) | 1139 | (776, 1865) | 0.0005 |

Data shown as median (25th, 75th percentile), statistical comparisons by Mann-Whitney U tests. CML, carboxymethyl lysine; FGF, fibroblast growth factor; HGF, hepatocyte growth factor; LMWF, low molecular weight fluorophore; PLGF, placental growth factor; sRAGE, soluble receptor for advanced glycation end-products; Tie-1 and Tie-2 are angiopoietin receptors 1 and 2, respectively; VEGF, vascular endothelial growth factor; VEGFR, vascular endothelial growth factor receptor.

Recent myocardial infarction was associated with reduced microvasculature density in myocardium remote from the site of infarction and alteration in plasma levels of AGEs and angiogenesis markers.

Those skilled in the art will appreciate that the disclosure described herein is susceptible to variations and modifications other than those specifically described. It is to be understood that the disclosure includes all such variations and modifications. The disclosure also includes all of the steps, features, compositions and compounds referred to, or indicated in this specification, individually or collectively, and any and all combinations of any two or more of the steps or features.

### BIBLIOGRAPHY

Anversa and Sonneblick Prog Cardovasc Dis 33:49-70, 1990
Berry et al., Eur Heart K 28:278-291, 2007
Black et al., J Hypertens 19:785-794, 2001
Goldstein et al., N Engl J Med 343:915-922, 2000
Gordon and Sweets Am J Pathol 12:545-552, 1936
Grundy et al., Circulation 112:2735-2752, 2005
Heusch et al., Circulation 120:1822-1836, 2009
Koerselman et al., Circulation 107:2507-2511, 2003
Levey et al., Ann Intern Med 130:461-470, 1999
Lim et al., Pediatr Res 60:83-87, 2006
Lloyd-Jones et al., Circulation 121:948-954, 2010
Messer et al., J Clin Invest 41:725-742, 1962
Naghavi et al., Circulation 108:1664-1672, 2003
Tomita et al., Hypertension 32:273-279, 1998
van Royen et al., J Am Coll Cardiol 55:17-25, 2009
Wallace et al., Diabetes Care 27:1487-1495, 2004

1. An assay to stratify a subject with cardiovascular disease (CAD) with respect to the risk of the subject developing a myocardial infarction (MI), said assay comprising:
(i) selecting a subject having symptoms of CAD or who is at risk of developing CAD; and
(ii) determining the plasma levels of an advanced glycation end (AGE) product or angiogenesis factor selected from the list consisting of carboxymethyl lysine (CML); low molecular weight fluorophore (LMWF); vascular endothelial growth factor-A (VEGF-A); vascular endothelial growth factor receptor-1 (VEGFR-1); angiopoietin-2:Tie-2; endostatin; placental growth factor (PLGF); and hepatocyte growth factor (HGF);
wherein the subject is stratified as having a MI or is at risk of developing a MI when:
(i) levels of one or more of CML, LMWF, VEGF-A, and/or Tie-2 are reduced compared to a control not having a MI; and/or
(ii) levels of one or more of VEGFR-1, VEGFR-1:VEGFA ratio, angiopoietin-2, angiopoietin-2:Tie-2 ratio, endostatin, PLGF and/or HGF are elevated compared to a control not having a MI.

2. The assay of claim 1 wherein the levels of two or more of the AGE products or angiogenesis factors are determined.

3. The assay of claim 1 wherein the levels of three or more of the AGE products or angiogenesis factors are determined.

4. The assay of claim 1 wherein the subject is a human subject.

5. A method of treatment or prophylaxis of a subject with cardiovascular disease (CAD), said method comprising stratifying the subject with respect to the risk of the subject developing a myocardial infarction (MI), said stratification comprising:
(i) selecting a subject having symptoms of CAD or who is at risk of developing CAD; and
(ii) determining the levels of an advanced glycation end (AGE) product or angiogenesis factor selected from the list consisting of carboxymethyl lysine (CML); low molecular weight fluorophore (LMWF); vascular endothelial growth factor-A (VEGF-A); vascular endothelial growth factor receptor-1 (VEGFR-1); angiopoietin-2:Tie-2; endostatin; placental growth factor (PLGF); and/or hepatocyte growth factor (HGF); and/or
wherein the subject is stratified as having a MI or is at risk of developing a MI when:
(i) levels of one or more of CML, LMWF, VEGF-A, and/or Tie-2 are reduced compared to a control not having a MI; and/or
(ii) levels of one or more of VEGFR-1, VEGFR-1:VEGFA ratio, angiopoietin-2, angiopoietin-2:Tie-2 ratio, endostatin, PLGF and/or HGF are elevated compared to a control not having a MI;
and then providing a therapeutic or behavoral modification to mitigate the risk of the MI.

6. The method of claim 5 wherein the subject is a human.

7. A method for diagnosing the likelihood of a subject exhibiting reduced coronary microvascular density and thereby at risk of having reduced coronary vasodilator reserve of myocardium remote from the site of an infarction, said method comprising:
(i) selecting a subject having symptoms of cardiovascular disease (CAD) or who is at risk of developing CAD; and
(ii) determining the levels of an advanced glycation end (AGE) product or angiogenesis factor selected from the list consisting of carboxymethyl lysine (CML); low molecular weight fluorophore (LMWF); vascular endothelial growth factor-A (VEGF-A); vascular endothelial growth factor receptor-1 (VEGFR-1); angiopoietin-2:Tie-2; endostatin; placental growth factor (PLGF) and hepatocyte growth factor (HGF);
wherein the subject is stratified as having reduced coronary microvascular density or is at risk of developing same when:
(i) levels of one or more of CML, LMWF, VEGF-A, and/or Tie-2 are reduced compared to a control not having a MI; and/or
(ii) levels of one or more of VEGFR-1, VEGFR-1:VEGFA ratio, angiopoietin-2, angiopoietin-2:Tie-2 ratio, endostatin, PLGF and/or HGF are elevated compared to a control not having a MI.

8. The method of claim 7 wherein the subject is a human.

9. A method of allowing a user to stratify a subject with cardiovascular disease (CAD) with respect to the risk of developing a myocardial infarction (MI), the method including:
(a) receiving data in the form of levels or concentrations of (i) an advanced glycation end (AGE) product or angiogenesis factor selected from the list consisting of one or more of carboxymethyl lysine (CML); low molecular weight fluorophore (LMWF); vascular endothelial growth factor-A (VEGF-A); vascular endothelial growth factor receptor-1 (VEGFR-1); angiopoietin-2:Tie-2; endostatin; placental growth factor (PLGF); and/or hepatocyte growth factor (HGF);
(b) processing the subject data via univariate or multivariate analysis to provide a risk index value;
(c) determining the status of the subject in accordance with the results of the risk index in comparison with predetermined values; and
(d) transferring an indication of the status of the subject to the user via the communications network;
wherein the status of the subject is that the subject is at risk of having MI or is at risk of developing a MI when:
(i) levels of one or more of CML, LMWF, VEGF-A, and/or Tie-2 are reduced compared to a control not having a MI; and/or
(ii) levels of one or more of VEGFR-1, VEGFR-1:VEGFA ratio, angiopoietin-2, angiopoietin-2:Tie-2 ratio, endostatin, PLGF and/or HGF are elevated compared to a control not having a MI.
